(19) United States
(12) Patent Application Publication
(10) Pub. No.: US 2024/0173246 A1
(43) Pub. Date: May 30, 2024

(54) SKIN BENEFIT COMPOSITIONS CONTAINING FERMENTED EXTACT OF A. MONTANA

(71) Applicant: Innovate90245, LLC, El Segundo, CA (US)

(72) Inventors: Stefani Germanotta, Maibu, CA (US); Gloria Ryu, Irvine, CA (US); Susan Goldsberry, Newport Coast, CA (US)

(73) Assignee: Innovate90245, LLC, El Segundo, CA (US)

(21) Appl. No.: 18/070,342

(22) Filed: Nov. 28, 2022

(51) Int. Cl. A61K 8/9789; A61K 36/287; A61Q 19/08

(52) U.S. Cl. CPC A61K 8/9789; A61K 36/287; A61Q 19/08; A61K 2800/522; A61K 2800/85

(57) ABSTRACT

Cosmetic, personal care or dermatologic composition comprised of an oil prepared by microbial fermentation A. montana and/or an aqueous fermentation broth produced from A. montana provide skin benefits by (a) reducing oxidative stress/free radical damage caused by exposure of the skin to an environmental stressor and (b) reducing the visible appearance of (i) redness/erythema, (ii) enlarged pores, (iii) dyschromia (uneven and/or blotchy skin tone), (iv) facial lines and wrinkles or other manifestations of photodamage.

### FIELD OF INVENTION

Cosmetic, personal care or dermatologic compositions; methods of providing one or more skin benefits by reducing the visible appearance of (i) redness/erythema, (ii) enlarged pores, (iii) dyschromia (uneven and/or blotchy skin tone), (iv) facial lines and wrinkles or other manifestations of photodamage.

### BACKGROUND OF INVENTION

Extracts of Arnica montana have been reported to possess antioxidant and anti-inflammatory properties as well as other therapeutic/pharmacologic activity including, antibacterial, antifungal, anti-rheumatic and analgesic immunomodulatory.

A variety of preparations and solubilities of A. montana extract are described in the published literature.

The 1990 edition of the International Cosmetic Ingredient Dictionary and Handbook describes a dark brown clear liquid prepared by the hydro-alcoholic maceration and percolation of the dried flower heads of A. montana that is soluble in water and insoluble in mineral oil.

U.S. Pat. No. 3,832,343 describes a multi-step process for preparing a water-soluble extract of A. montana and use of the resulting extract to improve circulation (reduce hematoma observed in skin bruising). First, plant material is combined at one or a mixture of petroleum ether, diethyl ether, acetone, chloroform, ethanol, and/or water. The resulting filtrate is treated with methanol and then ethyl acetate, yielding a concentrate of polar actives.

Since the mid-1990s, at least three companies have supplied Arnica extract in different carrier solutions/solubilizers. Ichimaru Pharcos Co., Ltd. offered solubilized flowers of A. montana in 1,3-butylene glycol. CLR (Chemisches Laboratorium Dr. Kurt Richter GmbH) supplied a yellow, oil-soluble mixture comprised of A. montana extract (1%-5%) in soybean (Glycine soja) oil (>50%), and tocopherol (<0.1%). Grau Aromatics GmbH & Co. supplied a water-soluble, clear, brown liquid Arnica montana Extract that is a mixture of 10%-25% flowers of A. montana in propylene glycol (>75%). The mixture is preserver with 0.6% Phenonip® (phenoxyethanol, methylparaben, butylparaben, ethylparaben, and propylparaben).

Among the over one hundred and fifty constituents which have been identified in A. montana, the following, alone and in combination, are reported to be functional activity (i.e., to impart or contribute to one or more skin benefit(s) as defined below: sesquiterpenes and sesquiterpene lactones (metacryl, isobutyryl, tygloyl, methacryloyl, isovaleryl, helenalin and 11alpha, 13-dihydohelenalin) and their short-chain carbonic acid esters; terpenes [monoterpenes, diterpenes, triterpenes (e.g., arnidiol)]; flavonoid glycosides—namely, glycosides of either flavones (apigenin, hispidulin and luteolin) or flavonols (kaempferol, quercetin); flavonoid glucuronides; flavonoid aglycones); coumarins (umbelliferone and scopoletin); phenolic acids (chlorogenic acid, caffeic acid, cynarine (hydroxycinnamic acid), dicaffeoyl quinic derivatives (1,3-3,5 and 4,5 dicaffeoyl quinic acids); and oligosaccharides; essential oils; carotenoids; lignans; alkaloids (tussilagine and isotussilagine).

Monofunctional sesquiterpene lactones (having one α,β-unsaturated carbonyl moiety) are reported to have better penetration, and, therefore, increased bioavailability and increased efficacy at the site of treatment. See, Matos S et al, Pharmaceutics. 2021 Jun. 30; 13(7):991; see also, Huber R et al. Complement Therap Med 2011; 19: 276-280.

The anti-inflammatory properties of A. montana have been explained in terms of different mechanisms of action than non-steroidal anti-inflammatory drugs (e.g., acetyl salicylic acid)—namely, in terms of transcription factors for Nuclear factor-κB (NF-kB) and heat shock proteins (HSP), which mediate the inflammatory process. Helenalin, a sesquiterpene lactone in A, montana, selectively inhibits transcription factor NF-kB. Lyβ G. et al. B. Biol Chem 1997; 378: 951-961. Helenalin 2-methylbutyrate boosts HSP70 gene expression and activates the transcription factor for HSP-1, and has been described as having a hypopigmenting effect in cosmetics. Usui K et al. J Dermatol Sci 2015; 78: 67-75.

Compositions containing A. montana have been applied topically in forms ranging from creams, ointments and gels to tinctures and wet poultices are disclosed in the patent publications as well as scientific/technical literature:

U.S. Pat. No. 5,976,547 describes an analgesic/anti-inflammatory topical preparation comprised of a petroleum base or pluronic lecithin organogel containing A. montana extract (3-30%) in combination with one or more therapeutic or pharmaceutical agents, i.e. camphor, menthol, eucalyptus oil, mint oil, guaifenesin, topical analgesics, non-steroidal anti-inflammatory drugs or either transdermal opioid.

The 3^{rd} Edition of the European Pharmacopoeia describes a tincture comprised of one part A. montana flowers to ten parts ethanol. The resulting tincture has 0.04% of the sesquiterpene lactone hydrohelenalin tiglate.

US Pre-Grant Patent Application Publication 2009/0104292 describes the use of A. montana at a similar concentration (30-40%) in a similar vehicle (petrolatum base) to post-traumatic bruising of skin after a surgical procedure, including, laser treatment.

U.S. Pat. No. 5,795,573 describes a homoeopathic topical anti-inflammatory preparation containing synergistic combination of extracts from A. montana, Rhus toxicodendron (Atlantic poison oak) and Aesculus hippocastanum (Horse chestnut) and Belladonna.

Use of A. montana to improve micro-circulation and potentiate absorption and therapeutic benefits from other active ingredients is described, for example, in U.S. Pat. No. 4,569,839 which is directed to more efficacious treatment and protection of skin by topical composition comprised of Vitamin E and Arnica oil.

### SUMMARY OF THE INVENTION

### Technical Problem

There has been and remains a long-felt but unmet need for topically-applied ingredients that reduce oxidative stress/free radical damage caused by exposure of the skin to an environmental stressor [UV radiation, High Energy Visible light (commonly known as “blue light”, emitted, for example, from electronic devices such as computers and smartphones) and atmospheric pollutants (airborne particulates)]. Additionally, there has been and remains a long-felt but unmet need for topically-applied ingredients that reduce the visible appearance of (i) redness/erythema, (ii) enlarged pores, (iii) dyschromia (uneven and/or blotchy skin tone), (iv) facial lines and wrinkles or other manifestations of photodamage.

### Technical Solution

Cosmetic, personal care or dermatologic composition comprised of an oil prepared by microbial fermentation A. montana and/or an aqueous fermentation broth produced from A. montana provide skin benefits by (a) reducing oxidative stress/free radical damage caused by exposure of the skin to an environmental stressor and (b) reducing the visible appearance of (i) redness/erythema, (ii) enlarged pores, (iii) dyschromia (uneven and/or blotchy skin tone), (iv) facial lines and wrinkles or other manifestations of photodamage.

### DETAILED DESCRIPTION OF THE INVENTION

Fermented Arnica oil is prepared by a microbial fermentation process, which in certain embodiments includes the steps of drying an extract of A. montana into a powder, pulverizing the powder, combining the powder with a bacterial culture containing one or more bacteria from one of the following genera: Lactobacillus; Bacillus; Bifidobacterium.

Fermented Arnica water is an aqueous fermentation broth produced from A. montana.

As will be appreciated by the personal having ordinary skill in the arts of botanical chemistry and topical formulation, constituents in the fermented Arnica oil and/or fermented Arnica water used in compositions and can and will vary by plant part (flower, seeds, rhizomes or roots), soil and climate conditions in different geographies, extraction techniques, and processing including selection of bacteria.

Fermented oils of A. montana that are used to formulate compositions of the present invention, and methods of using compositions containing fermented oils of A. montana can, and in certain preferred embodiments, do contain (a) an essential oil comprised of fatty acids, thymol derivatives, monoterpenes and/or sesquiterpenes and/or (b) a carotenoid.

As used in the present disclosure, a botanical antioxidant extract is a plant-derived ingredient that can reduce oxidative damage or quench free radicals as measured by FRAP assay or DPPH assay, both known to the person having skill in the art. I Benzie and J J Strain, Anal. 25 Biochem. Vol. 239 No. 1, pp. 70-76 (1996) (FRAP); V Bondet et al, Food Sci Technol, Vol. 30, pp. 609-615 (1997) (DPPH). Other methods known to the skilled artisan that can be used to measure antioxidant capacity are described by R L Prior et al, J. Agric. Food Chem. Vol. 53, pp. 4290-4302 (2005) (oxygen radical absorbance capacity assay, Trolox equivalent antioxidant capacity assay).

Skin benefits achieved by use of compositions containing a fermented Arnica oil and/or a fermented Arnica water of the present invention may be quantified using one or more of the following instrumental measurements, all of which are known to the skilled artisan:
(i) Reduction in trans-epidermal water loss (Tewameter)
(ii) Increased skin hydration (Corneometer)
(iii) Improved skin barrier function (Evaporimeter)
(iv) Increased skin elasticity (Cutometer)
(v) More even skin tone (Chromameter)

The first three skin benefits—reduced trans-epidermal water loss, increased skin hydration, improved skin barrier function—can be expressed as decreased dryness as described below.

Use of composition containing a fermented Arnica oil and/or a fermented Arnica water of the present invention has one or more anti-aging skin benefits including reduction in the appearance of one or more of (i) redness/erythema, (ii) enlarged pores, (iii) dyschromia (uneven and/or blotchy skin tone), (iv) facial lines and wrinkles or other manifestations of photodamage.

Skin redness/irritation and skin dryness can be measured by an expert grader for using a four-point Visual Analogue Scale (VAS). Redness/Irritation is graded on the following VAS: 0=no to very little erythema, slight scaling; 1=weak erythema, possibly slight infiltration; 2=marked erythema, infiltration, possibly vesicles and crusting; 3=pronounced erythema, infiltration, possibly vesicles, bullae, pustules and/or pronounced crusting. Skin dryness is graded from 0=no evidence of dryness to 4=severe flaking, peeling and/or fissures.

Clinical photography can be used to measure reduction in the appearance of facial fine lines and wrinkles using image analysis software, for example VISIA® from Canfield Scientific.

1. A topically-applied composition comprised of a fermented extract of Arnica montana.

2. The topically-applied composition of claim 1 wherein the fermented extract of Arnica montana is an oil prepared by microbial fermentation of Arnica montana

3. The topically-applied composition of claim 1 wherein the fermented extract of Arnica montana is an aqueous broth produced by fermentation of Arnica montana.

4. The topically-applied composition of claim 1 wherein the fermented extract of Arnica montana contains a sesquiterpene lactone.

5. The topically-applied composition of claim 1 wherein the fermented extract of Arnica montana contains a bioflavonoid or phenol.
